(19) United States
(12) Patent Application Publication
(10) Pub. No.: US 2022/0056518 A1
(43) Pub. Date: Feb. 24, 2022

(54) APPARATUS AND METHOD FOR HIGH THROUGHPUT PARALLEL NUCLEIC ACID SEQUENCING

(71) Applicant: ARDENT BIOMED GUANGDONG CO., LTD., Guangzhou (CN)

(72) Inventor: David Xingfei DENG, Mountain View, CA (US)

(21) Appl. No.: 17/299,247

(22) PCT Filed: Apr. 19, 2019
(86) PCT No.: PCT/CN2019/083381

§ 371 Date: Jun. 2, 2021

(51) Int. Cl. C12Q 1/6869; G01N 27/414

(52) U.S. Cl. CPC C12Q 1/6869; G01N 27/4145

(57) ABSTRACT

A method and an apparatus for nucleic acid sequencing are provided. The method includes immobilizing capturing oligonucleotides with different sequences in reaction wells, immobilizing single-stranded nucleic acid templates in the reaction wells via annealing between the templates and the capturing oligonucleotides, amplifying the immobilized nucleic acid templates and producing a population of template clones annealed with a plurality of sequencing primers. The method further includes sequentially disposing different types of nucleotide trisphosphates, detecting, by ion-sensitive field-effect transistors, ion concentration change in the reaction wells in response to incorporation of one of the nucleotide trisphosphates at 3′ end of sequencing primers, when the nucleotide trisphosphates is complementary to a corresponding nucleotide in the template clones, and sequencing the template clones by repeating the sequentially disposing and the detecting. A method for producing single-stranded nucleic acid template clones on a reaction well array is also provided.

### FIELD OF THE TECHNOLOGY

The present disclosure relates generally to the field of nucleic acid sequencing. More particularly, the present disclosure relates to apparatus and method for nucleic acid sequencing.

### BACKGROUND OF THE DISCLOSURE

Nucleic acid sequencing includes determination of the order of nucleotides, the chemical building blocks that make up nucleic acid. In a typical arrangement, DNA or RNA samples are fragmented, enriched, sequenced and analyzed to obtain the sequence information, which could be utilized in a broad range of biological and pharmaceutical applications including diagnostics of genetic diseases, drug trials and pharmacogenomics, as well as other applications such as evolutionary biology and forensics. Especially since the completion of the Human Genome Project in 2001, a rapid expansion of knowledge about human DNA and genetic variation has been initiated, which further boosts the development of nucleic acid sequencing technologies.

A number of sequencing techniques have been developed and some of them are commercialized. One of them is based upon a fluorescent imaging platform. Each type of deoxynucleoside triphosphate (e.g., dATP, dCTP, dTTP and dGTP) is labeled with fluorescently labeled reversible terminators. During the sequencing process, each sequencing cycle only allows a single dNTP added to the growing oligonucleotide strand. Concurrently with the single dNTP incorporation, the fluorescently labeled reversible terminator is imaged to identify a corresponding base in the template strand, and the terminator is subsequently cleaved to allow the incorporation of next dNTP. This method requires a delicate fluorescent imaging platform as well as at least four dNTPs labeled with different fluorescently labeled reversible terminators as building blocks, which cause high instrument cost and more importantly, significant reagent cost leading to high expense per run, especially for sequencing with a large genome size.

Alternatively, ion semiconductor sequencing is another sequencing technique based on detection of hydrogen ions released from incorporation of dNTPs into a growing nucleotide strand. Hydrogen ions are natural byproducts of polymerase-catalyzed nucleotide extension reactions. When a single dNTP is incorporated into the growing nucleotide strand, it releases one hydrogen ion which can be detected by an ion-sensitive field-effect transistor to generate an electronic signal. If homopolymer repeats are present, multiple hydrogen ions are released, corresponding to proportional increase in the electronic signals. Prior to sequencing process in the ion semiconductor sequencing method, DNA templates are attached onto micrometer-sized beads which are compartmentalized into water-oil emulsion droplets containing PCR reaction mixture. In the aqueous water-oil emulsion, each of the droplets containing the micrometer-sized bead functions as a PCR microreactor that amplifies the attached DNA template fragments. However, emulsion PCR is a time-consuming process requiring multiple steps (forming and breaking emulsion, PCR amplification, enrichment, etc.). Further, the emulsion breaking and bead washing are usually carried out by centrifugation, during which the beads may aggregate causing sample loss. It is also relatively inefficient since only around two thirds of the emulsion microreactors actually contain one bead while other emulsion droplets are empty. Therefore, an extra step may be required to separate empty emulsion droplets leading to more potential inaccuracies.

The disclosed apparatus and method for nucleic acid sequencing are directed to solve one or more problems set forth above and other problems.

### SUMMARY

The present disclosure provides a method for nucleic acid sequencing. The method for nucleic acid sequencing may include immobilizing at least two capturing oligonucleotides with different sequences in a plurality of reaction wells; and immobilizing a plurality of single-stranded nucleic acid templates in the plurality of reaction wells via annealing between the plurality of single-stranded nucleic acid templates and the at least two capturing oligonucleotides, where each of the single-stranded nucleic acid templates includes two regions complementary to the different sequences of the at least two capturing oligonucleotides, respectively. The method for nucleic acid sequencing may further include amplifying the immobilized plurality of single-stranded nucleic acid templates and producing a population of single-stranded nucleic acid template clones on the surface of the plurality of reaction wells, where the population of the single-stranded nucleic acid template clones is annealed with a plurality of sequencing primers, sequentially disposing different types of nucleotide trisphosphates into the plurality of reaction wells where the different types of nucleotide trisphosphates are known, and detecting, by one or more ion-sensitive field-effect transistors (ISFETs), ion concentration change in the plurality of reaction wells in response to incorporation of one of the different types of nucleotide trisphosphates at 3′ end of the sequencing primer, when the one of the different types of nucleotide trisphosphates is complementary to a corresponding nucleotide in the population of single-stranded nucleic acid template clones, and sequencing the population of single-stranded nucleic acid template clones by repeating the sequentially disposing of the different types of nucleotide trisphosphates and the detecting, by the one or more ISFETs, of the ion concentration change in the plurality of reaction wells.

In one embodiment of the present disclosure, a number of the plurality of single-stranded nucleic acid templates immobilized on a surface of each of the reaction wells via the annealing may be less than or equal to a pre-determined value, where the pre-determined value may be one. In another embodiment, a total number of the plurality of single-stranded nucleic acid templates disposed into the plurality of reaction wells may be less than or equal to a total number of the plurality of reaction wells, for example, the total number of the plurality of single-stranded nucleic acid templates disposed into the plurality of reaction wells may be less than or equal to 70% of the total number of the plurality of reaction wells.

In another embodiment of the present disclosure, the amplifying the immobilized plurality of single-stranded nucleic acid templates and producing the population of single-stranded nucleic acid template clones on the surfaces of the plurality of reaction wells may further include amplifying the immobilized plurality of single-stranded nucleic acid templates, thereby generating a plurality of double-stranded nucleic acid template clones, denaturing the plurality of double-stranded nucleic acid template clones, and producing a population of single-stranded nucleic acid template clones on the surface of the plurality of reaction wells.

The method for nucleic acid sequencing may further include the step of determining a loading rate of the plurality of reaction wells, where the load rate may include a ratio between a number of the reaction wells containing the immobilized nucleic acid templates and a total number of the plurality of reaction wells. To improve the loading rate and sequencing efficiency, in another embodiment, the method for nucleic acid sequencing may further include repeating the step of disposing the plurality of single-stranded nucleic acid templates into the plurality of reaction wells and the step of amplifying the immobilized plurality of single-stranded nucleic acid templates, and the loading rate may be determined after each loading cycle including the step of disposing the plurality of single-stranded nucleic acid templates into the plurality of reaction wells and the step of amplifying the immobilized plurality of single-stranded nucleic acid templates.

In another embodiment, the method for nucleic acid sequencing may further include the step of disposing a plurality of microbeads into the plurality of reaction wells, where a surface of the plurality of microbeads may be attached with single-stranded oligonucleotides or polymers, and the step of disposing the plurality of microbeads into the plurality of reaction wells may be concurrently with, or before the step of detecting, by the one or more ISFETs, ion concentration change in the plurality of reaction wells.

The present disclosure also provides an apparatus for nucleic acid sequencing, including a sensor array, including a plurality of ion-sensitive field-effect transistors (ISFETs) configured to provide at least one output signal corresponding to a concentration or presence of one or more ions proximate thereto, and a flow cell including an input, an output and a flow chamber, where the flow chamber may be in fluidic connection with an opening of each reaction well of an array of reaction wells, where at least two capturing oligonucleotides with different sequences may be immobilized on a surface of each of the reaction wells, and the different sequences of the at least two capturing oligonucleotides are complementary to two regions of a to-be-sequenced nucleic acid template. The apparatus for nucleic acid sequencing may further include a fluidics delivering unit, configured to be in fluidic connection with the input of the flow cell, and configured to deliver at least one of the to-be-sequenced nucleic acid template and different types of known nucleotide trisphosphates, in a direction from the input to the output, to the reaction chamber. In one embodiment of the present disclosure, each of the reaction wells may be associated with one of the plurality of ISFETs in the sensor array, and the one of the plurality of ISFETs may be configured to provide the at least one output signal in response to ion concentration change in each of the reaction wells, the ion concentration change may correspond to incorporation of one of the different types of nucleotide trisphosphates at 3′ end of a sequencing primer annealed to the to-be-sequenced nucleic acid template, when the one of the different types of nucleotide trisphosphates is complementary to a corresponding nucleotide in the to-be-sequenced nucleic acid template.

In one embodiment of the present disclosure, the fluidics delivering unit may further be configured to deliver a plurality of microbeads into the reaction chamber, such that the plurality of microbeads may control the diffusion of ionic byproducts generated from the incorporation of one of the different types of nucleotide trisphosphates at the 3′ end of the sequencing primer. In another embodiment, the sensor array and the array of reaction wells may be integrated on a same semiconductor chip.

The present disclosure also provides a method for producing single-stranded nucleic acid template clones on a reaction well array, the method including the steps of providing the reaction well array including a plurality of reaction wells, where at least two capturing oligonucleotides with different sequences may be immobilized on a surface of each of the reaction wells, disposing a solution including a plurality of single-stranded nucleic acid templates into the plurality of reaction wells, and amplifying the immobilized plurality of single-stranded nucleic acid templates, thereby generating a plurality of double-stranded nucleic acid template clones, denaturing the plurality of double-stranded nucleic acid template clones and producing a population of single-stranded nucleic acid template clones on the surface of the plurality of reaction wells. In one embodiment, each of the single-stranded nucleic acid templates may include two regions complementary to the different sequences of the at least two primers, and the plurality of single-stranded nucleic acid templates may be immobilized on the surface of the plurality of reaction wells via annealing between the single-stranded nucleic acid templates and the at least two capturing oligonucleotides. In another embodiment, a number of the single-stranded nucleic acid templates immobilized on a surface of each the plurality of reaction wells via the annealing may be less than or equal to a pre-determined value, for example, the pre-determined value may be one. In another embodiment, a total number of the plurality of single-stranded nucleic acid templates in the solution may be less than or equal to a total number of the plurality of reaction wells, for example, the total number of the plurality of single-stranded nucleic acid templates in the solution may be less than or equal to 70% of the total number of the plurality of reaction wells.

The method for producing single-stranded nucleic acid template clones on the reaction well array may further include the step of determining a loading rate of the plurality of reaction wells, where the load rate may include a ratio between a number of the reaction wells containing the immobilized single-stranded nucleic acid templates and a total number of the plurality of reaction wells. The loading rate may be determined by measuring, by one or more ion-sensitive field-effect transistors (ISFETs) configured to provide at least one output signal in response to a concentration or presence of one or more ions proximate thereto, ion concentration change corresponding to the amplification of the immobilized plurality of single-stranded nucleic acid templates in the reaction wells, where the plurality of reaction wells is associated with the one or more ISFETs.

The method for producing single-stranded nucleic acid template clones on the reaction well array may further include repeating the step of disposing the plurality of single-stranded nucleic acid templates into the plurality of reaction wells and the step of amplifying the immobilized plurality of single-stranded nucleic acid templates. Accordingly, the loading rate may be determined after each loading cycle including the step of disposing the plurality of single-stranded nucleic acid templates into the plurality of reaction wells and the step of amplifying the immobilized plurality of single-stranded nucleic acid templates.

Other aspects of the present disclosure can be understood by those skilled in the art in light of the description, the claims, and the drawings of the present disclosure.

### BRIEF DESCRIPTION OF THE DRAWINGS

To describe the technical solutions in the embodiments of the present disclosure more clearly, the following briefly introduces the accompanying drawings used for describing the embodiments. Apparently, the accompanying drawings in the following description show merely some embodiments of the present disclosure, and a person skilled in the art may still derive other drawings from these accompanying drawings without creative efforts.

FIG. 1 illustrates a block diagram of an exemplary nucleic acid sequencing apparatus according to various embodiments of the present disclosure;

FIG. 2 illustrates a diagram of a portion of the fluidics delivering unit and the detecting unit in an exemplary nucleic acid sequencing apparatus according to various embodiments of the present disclosure;

FIG. 3 illustrates a diagrammatic work flow of single-stranded nucleic acid template amplification and sequencing performed by an exemplary nucleic acid sequencing apparatus according to various embodiments of the present disclosure;

FIG. 4 illustrates a diagrammatic work flow of single-stranded nucleic acid template amplification performed by an exemplary nucleic acid sequencing apparatus according to various embodiments of the present disclosure;

FIG. 5 illustrates a flow chart of digital capturing and amplification of single-stranded nucleic acid templates to produce nucleic acid template clones, performed by an exemplary nucleic acid sequencing apparatus according to various embodiments of the present disclosure;

FIG. 6 illustrates a diagrammatic work flow of nucleic acid template sequencing performed by another exemplary nucleic acid sequencing apparatus according to various embodiments of the present disclosure;

FIG. 7 illustrates a diagrammatic work flow of nucleic acid template amplification and sequencing performed by another exemplary nucleic acid sequencing apparatus according to various embodiments of the present disclosure; and

FIG. 8 illustrates the use of microbeads to control ion diffusion in response to the nucleic acid template sequencing according to various embodiments of the present disclosure.

### DETAILED DESCRIPTION

The following describes the technical solutions in the embodiments of the present disclosure with reference to the accompanying drawings. Apparently, the described embodiments are merely some but not all the embodiments of the present disclosure. Other embodiments obtained by a person skilled in the art based on the embodiments of the present disclosure without creative efforts shall fall within the protection scope of the present disclosure.

The present disclosure provides an apparatus for nucleic acid sequencing, configured to determine the sequence information of the nucleic acid (e.g., DNA or RNA) in a sample. The nucleic acid sequencing apparatus may be used to determine the sequences of individual genes, larger genetic regions (e.g., clusters of genes), full chromosomes, or whole genome of an organism. Further, the nucleic acid sequencing apparatus may also be used in RNA sequencing and methylation sequencing by identifying methylation patterns in the genome. The nucleic acid sequencing apparatus according to the present disclosure may function in a variety of manners depending upon different applications, including sequencing-by-synthesizing as well as other sequencing methods such as sequencing by ligation or pyrosequencing, which will not be limited in the present disclosure. In a typical arrangement which will be described in greater detail below according to the present disclosure, based on genome size of the nucleic acid in a sample and the amount of the sample available to be sequenced, the nucleic acid may be firstly fragmented, followed by 5′ and 3′ adaptor ligation and denaturing process to create a sample library containing single-stranded nucleic acid templates. Further, the sample library may be disposed into a reaction chamber of the nucleic acid sequencing apparatus, in which these single-stranded nucleic acid templates may further be amplified to create a population of single-stranded nucleic acid template clones, the sequences of which may be determined by the subsequent sequencing process.

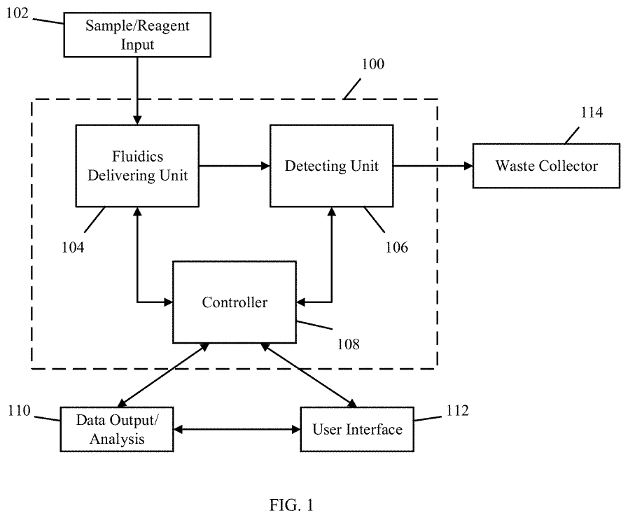

FIG. 1 illustrates a block diagram of an exemplary nucleic acid sequencing apparatus according to various embodiments of the present disclosure. The exemplary nucleic acid sequencing apparatus 100 may include a fluidics delivering unit 104, a detecting unit 106 and a controller 108. The fluidics delivering unit 104 may be configured to sequentially deliver nucleic acid sample solution to be sequenced, reagent solutions containing one or more of dNTPs, polymerase and electrolytes, as well as rinse or wash solutions to the detecting unit 106. The fluidics delivering unit 104 may include one or more of solution containers, valves, pumps and tubing configured to store and transfer solutions to the detecting unit 106 in a configurable manner. The detecting unit 106 may include one or more flow cells in fluidic connection with a plurality of micrometer-sized reaction wells. Each of the flow cells may include an input, an output and a reaction chamber. The input of the flow cell may be connected to the fluidics delivering unit 104, such that the solutions from the fluidics delivering unit 106 may be transferred into the reaction chamber through the input. The output of the flow cell may be connected to a waste collector 114. The reaction chamber may form a flow path in fluidic connection with openings of the plurality of reaction wells, such that the molecules flowing through the flow path, in a direction from the input to the output of the flow cell, may freely diffuse into the reaction wells, and further, the molecules in the reaction wells (e.g., excessive reagents) may diffuse out of the reaction well and exit from the output of the flow cell. In one embodiment, each of the reaction wells may have a sensor suitable for detecting characteristics of chemical or enzymatic reactions occurring within the reaction well, and convert the characteristics change to analog or digital signals as output. Optionally, a sample/reagent input 102 may be connected with the fluidics delivering unit, provide sample and reagent solutions to the fluidics delivering unit 102. In another embodiment, the detecting unit may further be connected to a waste collector 114 which contains reagent solutions and wash solutions exited from the flow cell of the detecting unit 106. More details regarding the fluidics delivering unit and the detecting unit will be described in greater detail below.

The controller 108 of the exemplary nucleic acid apparatus may be connected with the fluidics delivering unit 102 and the detecting unit 103. The controller 108 may include a general purpose or application-specific computer system configured to control the delivery of fluidics, acquire signals outputted from the detecting unit, process and output the output signals, as well as other functions as desired. For example, the controller may configure the order of the reagents being delivered to the detecting unit and preset the parameters of the fluidics delivery, including flow rate, flow duration, etc. The controller may also be configured to acquire and process signal outputted from the detecting unit 106 into formats recognizable by a data output/analysis unit 110, and a user interface 112.

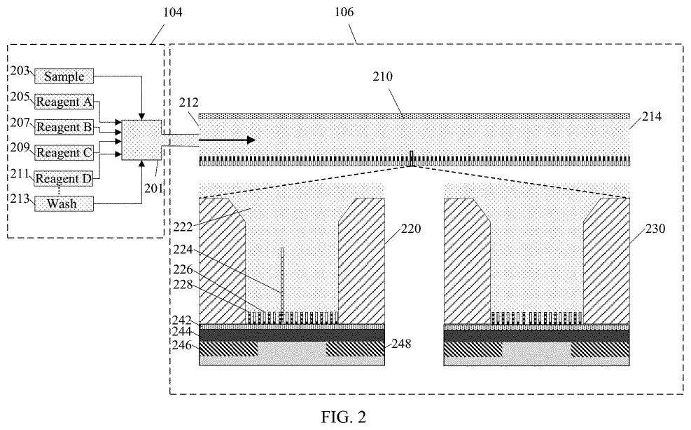

FIG. 2 illustrates a diagram of a portion of the fluidics delivering unit and the detecting unit in an exemplary nucleic acid sequencing apparatus according to various embodiments of the present disclosure. With reference to FIG. 2, The detecting unit 106 may include one or more flow cells. Each of the flow cells includes an input 212 in fluidic connection with the fluidics delivering unit 104, an output 214 in connection with the waste collector 114, and a flow chamber 210 in fluidic connection with opening portions of a plurality of reaction wells. For example, FIG. 2 illustrates an enlarged cross-sectional view of two reaction wells 220 and 230. The opening portion 222 of the reaction well 220 may be in fluidic connection with the flow chamber 210. In one embodiment, the flow cell may be a microfluidic device. The plurality of reaction wells may be disposed on a microfluidic chip made of semiconductors, polymers or metals by microfabrication or nanofabrication techniques known to one of ordinary skill in the art, for which the present disclosure will not described in detail herein. The microfluidic chip may further include a set of micro flow channels in connection with the reaction wells. Through the fluidic connection between the opening portions of the reaction wells and the micro flow channels, the molecules flowing through the reaction chamber may diffuse in and out of the reaction wells. For example, the plurality of reaction wells may be arranged in a matrix to form a reaction well array. In one embodiment, the microfluidic chip may include 10,000 to 1,000,000 micrometer-sized reaction wells, and the size of each reaction well may be below 20 micrometers. In another embodiment, the microfluidic chip may include 1,000,000 to 6,000,000 micrometer-sized reaction wells, and the size of each reaction well may be below 5 micrometers. It should be noted that the number, size, shape, volume or depth of the reaction wells in the reaction well array as well as the method used for manufacturing the reaction well array may be determined in accordance with various applications for which the present disclosure will not intend to be limiting.

In one embodiment, oligonucleotides (e.g. oligos) may be immobilized on the bottom portions of the reaction wells. These oligonucleotides may function as probes for capturing single-stranded nucleic acid templates in the sample solution flowing over the reaction wells, where each of the single-stranded nucleic acid templates may contain complementary sequences (e.g., adaptor sequences at 3′ and 5′ ends of the single-stranded nucleic acid templates) to the oligonucleotide probes. In one embodiment, at least two capturing oligonucleotides with different sequences may be immobilized on the bottom portion of the reaction wells, and the sequences of the capturing oligonucleotides may be complementary to different regions of the nucleic acid templates. With reference to FIG. 2, for example, two types of capturing oligos 226 and 228 may be immobilized on the bottom portions of the reaction wells 220 and 230. The sequence of the capturing oligo 228 may be complementary to adaptor sequence at one end of the single-stranded nucleic acid template 224, while the sequence of the capturing oligo 226 being complementary to the adaptor sequence at the other end of the template. Through the annealing between the capturing oligos and the nucleic acid template flowing over and diffusing into the reaction wells, the single-stranded nucleic acid template may be captured by the capturing oligos and therefore, immobilized onto the bottom portions of the reaction wells. It should be noted that although the oligos are immobilized on the bottom portions of the reaction wells as described above, the oligos may be immobilized on other solid surface (e.g., side walls) of the reaction wells for which the present disclosure will not intend to be limiting. The immobilization of the capturing oligos may be realized by conventional techniques used in DNA microarray fabrication, including spotting or printing methods in which the capturing oligos may be firstly synthesized and subsequently immobilized through mechanical deposition, as well as in situ synthesis of the capturing oligos on a solid support using photolithography.

In one embodiment, only one single-stranded nucleic acid template may be captured by the capturing oligos and immobilized in each reaction well. Subsequently, the single-stranded nucleic acid template may be amplified to generate a dense amount of nucleic acid template clones on the bottom portions of the reaction wells. In one embodiment, the immobilized capturing oligos may function as primers during the amplification process, therefore, the density of the capturing oligos may affect the density of the generated template clones. With reference to FIG. 2, for example, the reaction well 220 may contain a nucleic acid template strand 224. When the density of the immobilized capturing oligos was substantially low, a majority of the reaction wells may remain empty without any nucleic acid template after the sample solution containing the template was flushed over the reaction wells (e.g. the reaction well 230), which may reduce the efficiency of the template capturing, amplification and the following sequencing. On the other side, when the density of the immobilized capturing oligos was substantially higher, the generated template clones may be too dense at the bottom portions of the reaction wells, which may negatively affect the accuracy and efficiency of the following sequencing step.

With further reference to FIG. 2, each of the reaction wells may be connected to at least one sensor such that one or more of the characteristics of chemical or enzymatical reactions occurring within the reaction wells may be detected and measured by the at least one sensor. For example, the detecting unit 106 may include a sensor array including a plurality of sensors. Each of the sensors in the sensor array may correspond to a single reaction well in the reaction well array. In one embodiment, the sensors in the sensor array may be chemical field-effect transistor (ChemFET) measuring concentration change of one or more chemicals (e.g. a reactor or a product) in a reaction solution. In another embodiment, the sensor may be an ion-sensitive field-effect transistor (ISFET) detecting ion concentration change in a reaction solution. As shown in FIG. 2, for example, the ISFET sensors may be located below the reaction wells 220/230. In particular, the ISFET sensor may include a source electrode 246, a drain electrode 248, an ion-sensitive layer 242 and optionally a gate electrode 244. The ion-sensitive layer 242 may be in fluidic connection with the reaction solution disposed in the reaction wells and detect the change in ion concentration on the surface of or in proximity to the ion-sensitive layer, which may cause a change in the current between the source electrode 246 and the drain electrode 248. The current change corresponding to the ion concentration change within the reaction well may be directly outputted or further converted to voltage or impedance change as output signal, depending upon specific applications for which the present disclosure will not describe herein. It should be noted that the configurations of the ISFET with top-gate structure in FIG. 2 are for illustrative purposes only, any other suitable structures such as bottom-gate structures may be encompassed within the present disclosure. In other embodiments, the ISFET may have other configurations based upon specific applications for which the present disclosure will not limit herein.

In accordance with the aforementioned embodiments, the ISFET sensor in the exemplary sequencing apparatus may further include a reference electrode in fluidic connection with the reaction wells, providing a same voltage to all of the reaction wells. In one embodiment, the reference electrode may be one or more micro electrodes integrated on the sensor array. The sensor array may be fabricated on a circuit-connected substrate where the circuit is connected to the controller 104. As such, the signal change detected by each sensor may be collected and processed by the controller 104. In one embodiment, the sensor array and the reaction well array may be integrated on a same semiconductor chip.

In one embodiment, the sensor in the exemplary nucleic acid apparatus may be a pH-sensitive ISFET detecting concentration change of the hydrogen ions. For example, the pH-sensitive ISFET may detect the generation of hydrogen ions from a polymerase-catalyzed oligonucleotide extension reaction within the reaction wells. In particular, with each dNTP incorporating into a growing nucleotide strand, a hydrogen ion, as a natural byproduct of the dNTP incorporation, will be generated and diffused in proximity to the ion-sensitive layer of the ISFET and detected by the ISFET. Accordingly, the concentration of the released hydrogen ions may be proportional to the concentration of the incorporated dNTP. In another embodiment, four types of dNTPs may be sequentially delivered to the reaction wells in a pre-determined order, one type of dNTP at a time. In the presence of a primer annealed to the nucleic acid template to form a primer-template duplex in the reaction wells as well as a DNA polymerase, only a dNTP complementary to a next base in the template strand will be incorporated at 3′ end of the primer strand and release a hydrogen ion which may be detected by the ISFET and generate a positive signal output. The other three types of dNTPs which are not complementary to the based may be flushed out of the reaction well without generating a positive signal. Based on the positive signal generated with the incorporation of the complementary dNTP, the base on the template strand may be identified. When this reaction cycle is repeated, the sequence of the entire nucleic acid template may be identified in such sequencing-by-synthesizing manner.

With regard to the fluidics delivering unit 104, it may include one or more of solution containers, valves, pumps and tubing, for storing and transferring solutions to the detecting unit 106 in a configurable manner. As shown in FIG. 2, for example, the fluidic delivering unit 104 may include a valve unit 201 with pumps and tubing, configured to deliver sample solution stored in a sample solution container 203, as well as reagent A (e.g., a solution containing dATP, DNA polymerase and electrolyte) stored in reagent container 205, reagent B (e.g., a solution containing dGTP, DNA polymerase and electrolyte) stored in reagent container 207, reagent C (e.g., a solution containing dTTP, DNA polymerase and electrolyte) stored in reagent container 209, reagent D (e.g., a solution containing dCTP, DNA polymerase and electrolyte) stored in reagent container 211 and wash solution (e.g., weakly buffered solution) stored in wash solution container 213. In some of the embodiments, a portion or all of the reagent solutions and wash solutions may be integrated into one or more cartridges which could be installed into the exemplary nucleic acid apparatus.

Under the control of the controller 108, and the delivered solutions may vary depending upon the working stages of the nucleic acid sequencing apparatus. For example, during a sequencing-by-synthesis stage as described in the aforementioned embodiment, the delivered solution may sequentially deliver reagents (e.g., reagents A, B, C and D), followed by a washing solution. That is, the four types of dNTPs, one at a time, may be sequentially delivered to the reaction chamber of the detecting unit in the pre-determined order, e.g., dATP, dGTP, dTTP, dCTP, dATP, dGTP, dTTP, dCTP and so forth. After each delivery of single type of dNTP, the reaction chamber may be exposed with wash solutions to remove excessive dNTP. Optionally, a dNTP-destroying solution (e.g., apyrase), after the washing, may be delivered to eliminate any residual dNTP remaining in the reaction chamber and reaction wells. It should be noted that the aforementioned order of the dNTP addition is for exemplary purposes only, for which the present disclosure will not intend to be limiting. Optionally during the sequencing-by-synthesizing stage, the fluidics delivering unit 104 may further deliver a plurality of microbeads into the reaction chamber, such that the plurality of microbeads control diffusion of ionic byproducts generated from the incorporation of dNTPs at 3′ end of the sequencing primer, thereby enhancing the sensitivity of the ion detection when using an ISFET sensor.

In another embodiment, during a sample loading stage, the single-stranded nucleic acid templates may be delivered to the reaction chamber such that the template may be captured onto the surface of the reaction well for subsequent amplification. A number of the plurality of single-stranded nucleic acid templates immobilized on the surface of each of the reaction wells via the annealing may be less than or equal to a pre-determined value. In one embodiment, the pre-determined value may be one, that is, a single nucleic acid template may be immobilized on the surface of each reaction well. To realize the single nucleic acid template immobilization, in one embodiment, the fluidics delivering unit 104 may deliver a sample solution containing a low concentration of nucleic acid template in a per-determined flow rate for pre-determined duration, such that a single nucleic acid template may diffuse into each of the reaction wells and immobilized. Additionally, during other working stages, the fluidics delivering unit 104 may deliver cleavage solution for cleaving the linkers on the capturing oligos, and denaturing solution (e.g., containing NaOH) for removing one strand of the double-stranded amplified nucleic acid templates to form a population of single-stranded nucleic template clones on the surface of the reaction wells.

The present disclosure also provides a method for nucleic acid sequencing. The method may include the following steps: providing a plurality of reaction wells associated with one or more ISFETs configured to provide at least one output signal in response to ions disposed in the plurality of reaction wells, where at least two capturing oligonucleotides with different sequences may be immobilized on a surface of the plurality of reaction wells; disposing a plurality of single-stranded nucleic acid templates into the plurality of reaction wells, where each of the single-stranded nucleic acid templates may include two regions complementary to the different sequences of the at least two capturing oligonucleotides, and the plurality of single-stranded nucleic acid templates may be immobilized on the surface of the plurality of reaction wells via annealing between the plurality of single-stranded nucleic acid templates and the at least two capturing oligonucleotides; amplifying the immobilized plurality of single-stranded nucleic acid templates and producing a population of single-stranded nucleic acid template clones on the surface of the plurality of reaction wells, where the population of the single-stranded nucleic acid template clones may be annealed with a plurality of sequencing primers; sequentially disposing different types of nucleotide trisphosphates into the plurality of reaction wells, where the different types of nucleotide trisphosphates may be known; and detecting, by the one or more ISFETs, ion concentration change in the plurality of reaction wells in response to incorporation of one of the different types of nucleotide trisphosphates at 3′ end of the sequencing primer, when the one of the different types of nucleotide trisphosphates is complementary to a corresponding nucleotide in the population of single-stranded nucleic acid template clones; and sequencing the population of single-stranded nucleic acid template clones by repeating the step of sequentially disposing of the different types of nucleotide trisphosphates and the step of detecting, by the one or more ISFETs, the ion concentration change. The work flow of the exemplary method for nucleic acid sequencing will be described in greater detail below. It should also be noted that the exemplary sequencing method according to the present disclosure may be carried out by the exemplary apparatus for nucleic acid sequencing. Alternatively, the exemplary sequencing method according to the present disclosure may be carried out by other apparatus, for which the present disclosure will not intend to be limiting.

In one embodiment, the single-stranded nucleic acid template may firstly be pre-treated before disposing into the reaction wells. In particular, nucleic acid (e.g. genomic DNA) may be extracted and fragmented to generate a collection of double-stranded nucleic acid fragments of which the sequences may be of interest to obtain sequence information. After or concurrently with the fragmentation process, both 3′ and 5′ ends of the double-stranded nucleic acid fragments may be ligated with two adaptors, respectively, followed by a denaturing process to form a plurality of single-stranded nucleic acid templates, where each template may include two adaptors ligated at 3′ end and 5′ end, respectively. It should be noted that other sample preparation methods may also be used for which the present disclosure will not intend to limit. In one embodiment, the nucleic acid extraction and sample preparation may approximately take 90-120 minutes.

After the sample preparation process, the sample solution containing a plurality of single-stranded nucleic acid templates may be disposed into the fluidics delivering unit 104 through the sample/reagent input 102. The nucleic acid templates may flow in the reaction chamber and diffused into the opening portion of the plurality of reaction wells. In accordance with the aforementioned embodiments, capturing oligos with sequences complementary to the 3′ and 5′ end adaptors on the single-stranded template, respectively, may be immobilized on the bottom portion of the reaction wells. Through the annealing between the adaptor at 3′ or 5′ end of the template and the capturing oligo, the single-stranded template may be immobilized on the bottom portion of the reaction wells.

In one embodiment, a number of the plurality of single-stranded nucleic acid templates immobilized on the surface of each of the reaction wells via the annealing is less than or equal to a pre-determined value. For example, the pre-determined value may be one. In other words, for each of the reaction wells, it may be immobilized with a single nucleic acid template, alternatively it may contain none of the templates, resulting in digital capture of the nucleic acid templates, that is, either 1 or 0 single-stranded nucleic acid template may be immobilized in each reaction well. As shown in FIG. 2, with the sample solution containing the nucleic acid templates flowing in the reaction chamber, the reaction well 220 may be immobilized with a single nucleic acid template 224 and the reaction well 230 may be empty. The reaction well including a single template may be associated with a positive signal output (e.g. a binary readout of 1) while the reactions well including none of the templates may only show background signal configured as a negative signal output (e.g., a binary readout of 0). The digital capturing of the single-stranded nucleic acid template may possess a variety of advantages. For example, after the amplification of a single nucleic acid template in each reaction well, a dense cluster of template clones may be formed in the reaction well, bring in high reproducibility and precision to the following sequencing step. Additionally, the amplification of a single nucleic acid template through the digital capturing may realize absolute quantification without the need for running standard curves or reference, significantly improving the accuracy and sensitivity of the amplification and the sequencing step.

To realize the digital capturing of the nucleic acid template, in one embodiment, the concentration of the sample solution containing the templates may be properly adjusted to a pre-determined value. For example, the concentration of the template in the sample solution may be diluted so that a number of single-stranded nucleic acid templates per volume in the reaction chamber may be less than a total number of the reaction wells. In one embodiment, the number of the single-stranded nucleic acid templates per volume in the reaction chamber may be less than or equal to 90% of the total number of the reaction wells. Optionally, the number of the single-stranded nucleic acid templates per volume in the reaction chamber may be less than or equal to 80% of the total number of the reaction wells. Optionally, the number of the single-stranded nucleic acid templates per volume in the reaction chamber may be less than or equal to 70% of the total number of the reaction wells. In one embodiment, when the number of the nucleic acid templates per volume in the reaction chamber may be less than the total number of the reaction wells, the polyclonal capturing of the template into the reaction wells may be significantly reduced based upon Poisson distribution. That is, the ratio of the reaction wells immobilized with more than one single-stranded nucleic acid template to a total number of the reaction wells immobilized with nucleic acid templates may be reduced. For example, the polyclonal capturing of the nucleic acid template may be reduced to below 20%, when the number of the nucleic acid templates per volume in the reaction chamber may be ≤90%, ≤80% or ≤70% of the total number of the reaction wells. The low ratio of the polyclonal capturing of the template in the reaction wells may be removed during data analysis, such that it may not cause interference in the subsequent sequencing process.

Furthermore, to realize the digital capturing of the nucleic acid templates, the parameters of the fluidics delivering unit 104, e.g., the flow rate and duration of the sample solution containing the templates may also be adjusted to pre-determined settings. After the sample solution flows from the input of the flow path and filled the reaction chamber, it may be settled in the reaction chamber for a pre-determined duration, such that the template may diffuse in proximity to the capturing oligos immobilized in the reaction wells.

In another embodiment, the sample solution containing the single-stranded nucleic acid templates may be partitioned to generate a plurality of small droplets, each of the droplets including 1 or 0 of the templates. For example, a 20 microliter of sample solution may be partitioned into 20,000 nanoliter-sized droplets. Subsequently, these droplets may be injected into the fluidics delivering unit 104 through the sample/reagent input 102, flowing in the reaction chamber 210. With the concentration of the droplets in the reaction chamber as well as one or more of the flow parameters adjusted, a single droplet may be disposed into each of the reaction wells. The template within the single droplet may be released and hybridized with the capturing oligos in the reaction well to realize the digital capturing of the template.

The method for nucleic acid sequencing may further include the steps of amplifying the immobilized nucleic acid templates and producing a population of nucleic acid template clones on the surface of the plurality of reaction wells, where the population of the nucleic acid template clones is annealed with a plurality of sequencing primers; sequentially disposing different types of nucleotide trisphosphates into the plurality of reaction wells, where the different types of nucleotide trisphosphates are known; detecting, by the one or more ISFETs, ion concentration change in the plurality of reaction wells in response to incorporation of one of the different types of nucleotide trisphosphates at 3′ end of the sequencing primer, when the one of the different types of nucleotide trisphosphates is complementary to a corresponding nucleotide in the population of nucleic acid template clones; and sequencing the population of nucleic acid template clones by repeating the step of sequentially disposing of the different types of nucleotide trisphosphates and the step of detecting, by the one or more ISFETs, the ion concentration change. One or more embodiments in accordance with the aforementioned steps will be described as follows.

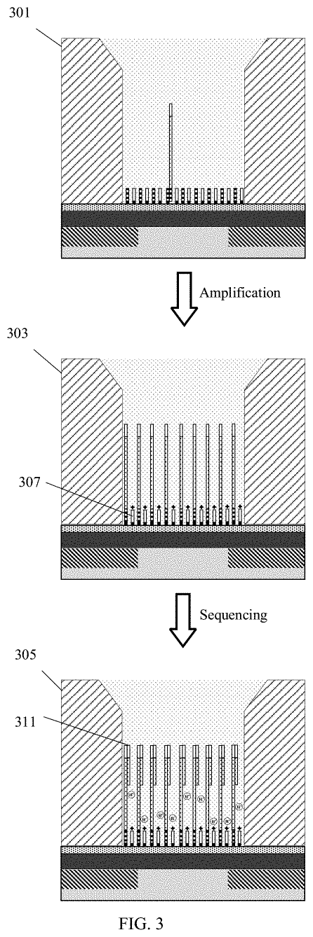

FIG. 3 illustrates a diagrammatic work flow of single-stranded nucleic acid template amplification and sequencing performed by an exemplary nucleic acid sequencing apparatus according to various embodiments of the present disclosure. After the digital capture of the template, a single nucleic acid template may be immobilized on the bottom portion of the reaction well 301. Subsequently, in the presence of amplification reagents containing DNA polymerase, dNTPs and electrolytes, the nucleic acid template may further be amplified to generate a plurality of nucleic acid template clones. For example, the nucleic acid template may be amplified by performing isothermal amplification in the presence of one or more DNA polymerases suitable for the specific amplification method and dNTPs as building blocks.

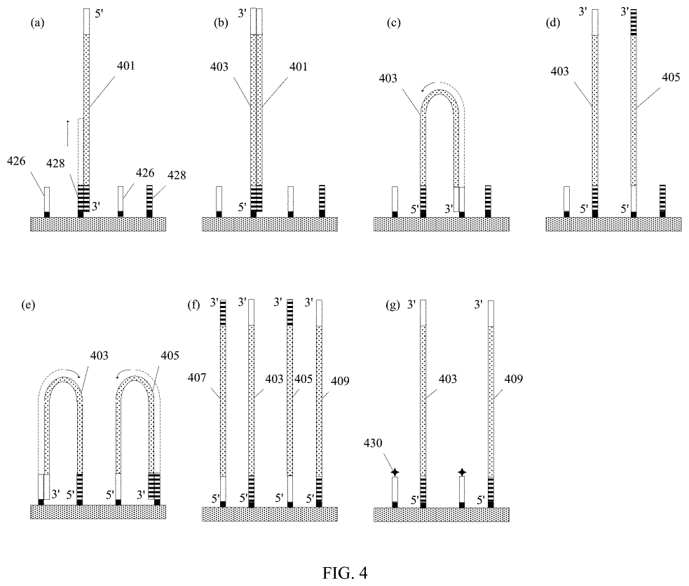

In one embodiment, bridge amplification method may be performed to amplify the single nucleic acid template on the bottom portion of each reaction well. FIG. 4 illustrates a diagrammatic work flow of single-stranded nucleic acid template amplification performed by an exemplary nucleic acid sequencing apparatus according to various embodiments of the present disclosure. In one embodiment with reference to step (a) of FIG. 4, a single nucleic acid template 401 including two adaptor sequences at 3′ and 5′ ends, respectively, may be captured by annealing to one of the capturing oligos 428 immobilized on the surface of the reaction well, where the adaptor sequences at 3′ end may be complementary to the sequence of the capturing oligo 428. The capturing oligo 428 may be extended in the presence of the nucleic acid template 401, DNA polymerase and dNTPs to form an elongated oligonucleotide strand 403 (see (b) of FIG. 4). After denaturing and removing of the original nucleic acid template 401, the extended elongated oligonucleotide strand 403 which may have a complementary sequence as the removed nucleic acid template 401, may bend over and approach the surface of the reaction well. The adaptor sequence at 3′ end of the oligonucleotide strand 403 may be complementary to and therefore, annealed to another capturing oligo 426. The capturing oligo 426 may extend using the oligonucleotide strand 403 as a template, to form an elongated oligonucleotide strand 405 having the same sequence as the nucleic acid template 401 (see (c) of FIG. 4). After denaturing (see (d) of FIG. 4), the two oligonucleotide strands 403 and 405 may further bend over and anneal to the capturing oligos 426 and 428, respectively, to form new oligonucleotide strands 407 and 409 using the capturing oligos as primers (see (e) and (f) of FIG. 4). The aforementioned steps may be rapidly repeated, thereby forming a dense amount of double-stranded nucleic acid template clones may be formed on the bottom of the reaction wells. For each amplification cycle, one capturing oligo immobilized in the reaction well may be occupied. With the completion of the bridge amplification, a majority of the capturing oligos may be occupied, resulting thousands of template clones in the reaction well.

In another embodiment of the present disclosure in accordance with (g) of FIG. 4, part of the amplified nucleic acid templates may be retained in the reaction wells, while the other part of the amplified nucleic acid templates may be cleaved and the corresponding capturing oligos and free 3′ ends of the template strands may be blocked. For example, the amplified nucleic acid template 403 and 409, having 5′ ends in proximity to the surface of the reaction wells and 3′ ends away from the surface of the reaction wells, may be retained in the reaction wells. The amplified nucleic acid template 405 and 407, which are the reverse strands of the template 403 and 409, may be cleaved, with corresponding capturing oligos being blocked by blocking reagents 430. As such, all of the amplified single stranded nucleic acid templates may have the same direction. Since the above amplification process starts with a single nucleic acid template, the precision and reproducibility of the amplification may be significantly improved, thereby increasing the accuracy of the following sequencing step. It should be noted that the bridge amplification method in the aforementioned embodiment is for exemplary purposes only. Other amplification methods in accordance with suitable DNA polymerase and other reagents used in specific protocols, for which the present disclosure will not intend to be limiting.

As described above, the digital capture of the template in the reaction well may also result in empty reaction wells containing none of the template. Accordingly, the digital capturing of the nucleic acid template within the reaction well may need to be monitored, thereby determining a loading rate of the reaction wells. In particular, the load rate may be a ratio between a number of the reaction wells containing the immobilized nucleic acid templates and a number of all of the reaction wells. In one embodiment, when an ISFET sensor was associated with each of the reaction wells, the amplification of the nucleic acid template may also be monitored by output signals of the ISFET sensors in response to ion concentration change during the template amplification. The loading rate may further be determined by monitoring the output signals of the ISFET sensors during the amplification of the nucleic acid template. For example, the sample solution containing the single-stranded nucleic acid templates may be firstly flushed into the reaction chamber for digital capturing of the template in each of the reaction wells. When a single template is immobilized in one of the reaction wells, the ISFET sensor associated with the reaction well may be configured to monitor the amplification process of the single template by measuring the concentration change of the released hydrogen ions during the amplification, and outputting signals (e.g. a binary signal of one). For empty reaction wells without nucleic acid template, the associated ISFET sensors may only show background signal as a negative output signal (e.g. a binary output of zero). As such, the loading rate of the reaction wells may be determined by quantifying the number of reaction wells with a binary output of one.

In accordance with the aforementioned embodiments, the present disclosure also provides a method for producing single-stranded nucleic acid template clones on a reaction well array, including the steps of: providing the reaction well array including a plurality of reaction wells, where at least two capturing oligonucleotides with different sequences may be immobilized on a surface of each of the reaction wells; disposing a solution including a plurality of single-stranded nucleic acid templates into the plurality of reaction wells, where each of the single-stranded nucleic acid templates may include two regions complementary to the different sequences of the at least two capturing oligonucleotides, the plurality of single-stranded nucleic acid templates may be immobilized on the surface of the plurality of reaction wells via annealing between the single-stranded nucleic acid templates and the at least two capturing oligonucleotides, and a number of the single-stranded nucleic acid templates immobilized on the surface of each the plurality of reaction wells via the annealing may be less than or equal to a pre-determined value; and amplifying the immobilized plurality of single-stranded nucleic acid templates and producing a population of double-stranded nucleic acid template clones on the surface of the plurality of reaction wells. By denaturing the plurality of double-stranded nucleic acid template clones and a population of single-stranded nucleic acid template clones may be produced on the surface of the plurality of reaction wells. It should also be noted that the exemplary method for producing nucleic acid template clones on a reaction well array according to the present disclosure may be carried out by the exemplary apparatus for nucleic acid sequencing. Alternatively, the exemplary method may be carried out by other instruments or performed on other platforms, for which the present disclosure will not intend to be limiting.

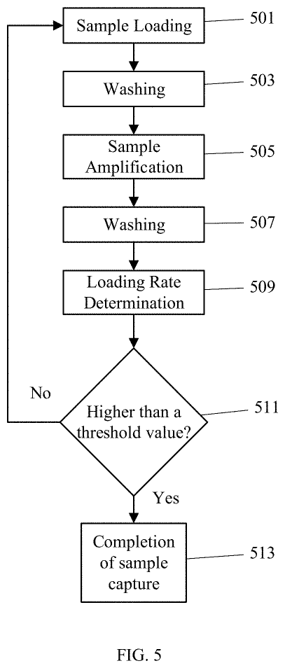

FIG. 5 illustrates a flow chart of digital capturing and amplification of single-stranded nucleic acid templates to produce nucleic acid template clones, performed by an exemplary nucleic acid sequencing apparatus according to various embodiments of the present disclosure. For example, a loading cycle for digital capturing of the single-stranded nucleic acid template may include step 501 of sample loading and step 505 of sample amplification, followed by washing steps 503 and 507, respectively. During step 505, the sample amplification may be monitored, thereby determining a loading rate for the currently loading cycle in step 409. When the loading rate was lower than a pre-determined threshold value, the loading cycle may be repeated, until the determined loading rate is higher than or equal to the threshold value, the loading may be completed and ready for sequencing. In particular, during the step 501 of sample loading, the sample solution containing a pre-determined concentration of nucleic acid template may be disposed into the reaction chamber through the fluidic delivering unit 104, and flushed out by the wash solution during the step of 503. A portion of the reaction wells may be immobilized with a single nucleic acid template while another portion of the reaction wells may still be empty without any template. In accordance with the aforementioned embodiments, the nucleic acid template may be amplified during the sample amplification step 505 and monitored for the amplification process, followed by the washing step 507. Concurrently with or after the amplification step 505, a loading rate corresponding to the current loading cycle may be determined (step 509) and compared with the pre-determined threshold value (step 511). In one embodiment, the production of nucleic acid template clones on a reaction well array may approximately take 40-70 minutes.

In accordance with the aforementioned embodiments, the loading rate may be determined in different ways. For example, the ISFETs may be configured to provide at least one output signal in response to ions disposed in the plurality of reaction wells, where the ion concentration change may correspond to the amplification of the immobilized plurality of nucleic acid templates in the reaction wells associated with the ISFETs.

The loading cycle may be repeated until the loading rate exceeds the pre-determined threshold value to indicate completion of sample capture (see e.g., 513 of FIG. 5). In one embodiment, the loading step and the amplification step may be repeated twice, that is, loading→amplifying→loading→amplifying. Optionally, the loading cycle including the loading step and amplification step may be repeated three times or more. Additional washing steps may be needed between two loading cycles. The pre-determined threshold value and cycle numbers may be pre-set by an operator through the user interface 112. Furthermore, the concentration of the template in the sample solution used for each loading cycle may be the same. Optionally, the fluidics delivering unit may deliver a small portion of the sample solution in a configurable manner while the remaining sample solution stored in the sample solution container 204 and ready to be delivered for next loading cycle. Alternatively, the concentration of the nucleic acid template in the sample solution used for the initial loading cycle may vary from the subsequent loading cycles.

In accordance with the aforementioned embodiments and FIG. 3, with the completion of amplification of the single-stranded template in the reaction wells, a dense amount of double-stranded nucleic acid templates may be formed in the bottom portions of the reaction wells. After denaturing the duplex and removing reverse strands, a plurality of single-stranded templates may remain immobilized in the reaction wells and ready for sequencing, while other oligos blocked by the blocking molecules 307. As shown in FIG. 3, the nucleic acid template may be enriched on the bottom portions of the reaction well 303 and ready for sequencing. During the sequencing step, in the presence of sequencing primers annealed to the nucleic acid template clones, DNA polymerase and known dNTPs, by detecting one or more characteristics of the sequencing primer extension reaction, the incorporated dNTPs at 3′ end of the growing sequencing primer may be identified in the sequencing-by-synthesis manner, as described below in greater details.

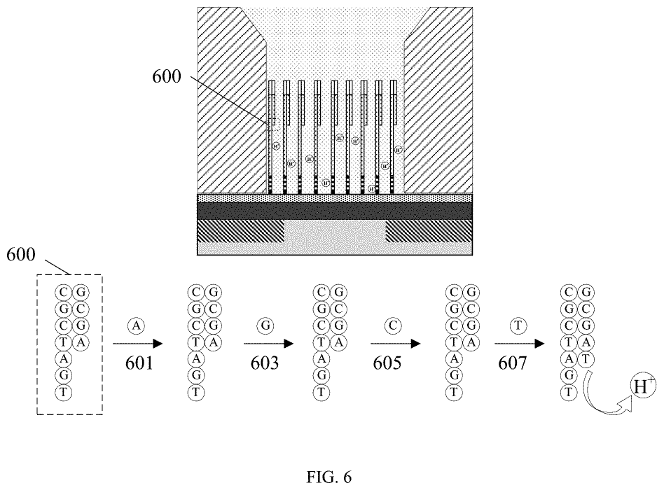

FIG. 6 illustrates a diagrammatic work flow of nucleic acid template sequencing performed by another exemplary nucleic acid sequencing apparatus according to various embodiments of the present disclosure. In particular, the fluidics delivering unit 104 may sequentially deliver a single type of dNTP into the reaction chamber in a pre-determined order, for example, in the order of dATP, dGTP, dCTP and dTTP. FIG. 6 also illustrates an enlarged portion of the growing duplex 600 during the sequencing step. The next base on the template strand with the sequence of CGCTAGT waiting for polymerization is A. With the sequential addition of non-complementary dATP, dGTP and dCTP in steps 601, 603 and 605, respectively, the next base A at 3′ end of the sequencing primer remain unpolymerized. With the addition of complementary dTTP in step 607, a dTTP may be incorporated into the growing sequencing primer, accompanied with the release of a hydrogen ion as byproduct.

In accordance with the aforementioned embodiments, when each of the sensor well is connected with an ISFET sensor, the ion-sensitive layer of the ISFET sensor may detect and measure the concentration change of the hydrogen ions corresponding to the incorporation of the dNTPs at 3′ end of the sequencing primer (e.g., dTTP). In one embodiment, when a homopolymer region (e.g., poly(dA)) is present in the template, the incorporation of multiple dTTP molecules may result in a multi-fold signal change corresponding to the number of the repeatable bases in the template. For example, a homopolymer region including AA sequence repeats may cause a two-fold signal change compared to the signal generated by a single T in the template. The detection of hydrogen ions by the use of ISFET sensors may only require natural dNTPs, rather than dNTPs with different fluorescently labeled reversible terminators and complex fluorescent imaging platform, thereby significantly reducing the cost of the sequencing apparatus and reagent cost per run. Furthermore, the detection of hydrogen ions by the use of ISFET sensors may improve the sequencing efficiency. In one embodiment, the sequencing process may approximately take 60-90 minutes.

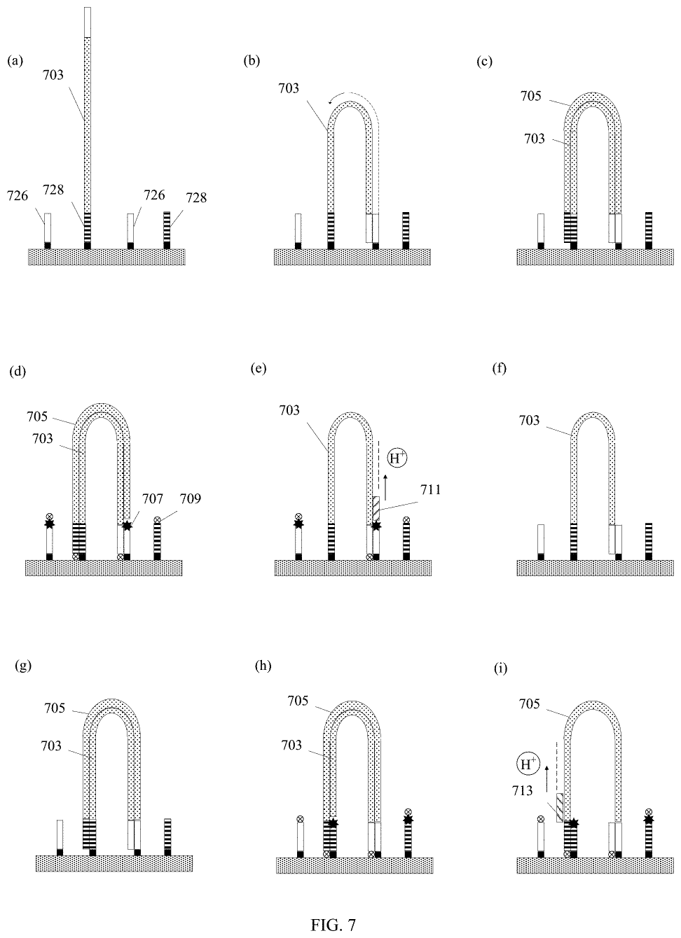

Alternatively, FIG. 7 illustrates a diagrammatic work flow of nucleic acid template amplification and sequencing performed by another exemplary nucleic acid sequencing apparatus according to various embodiments of the present disclosure. With reference to FIG. 7, the sequencing of the amplified nucleic acid template may be realized at both ends of the template sequence. For example, the nucleic acid template 703 immobilized on the surface of the reaction well may bend over and annealed to the capturing oligo 726 (see (a) and (b) of FIG. 7). The capturing oligo 726 may extend to generate an elongated oligonucleotide strand 705. These steps may be repeated, thereby generating a plurality of double-stranded nucleic acid clones (see (c) of FIG. 7). Subsequently, the strand 705 may be cleaved by the cleavage reagent 707 and washed away after the denaturing process. The ends of the oligonucleotide 703 as well as the capturing oligos 726 and 728 may be blocked by the addition of ddNTPs 709 (see (d) and (e) of FIG. 7). With the addition of a sequencing primer 511, the sequencing may be started from 3′ end of the nucleic acid template clone 703. With the extension of the sequencing primer 511 in the presence of DNA polymerase and dNTPs, the incorporation of each dNTP into the elongated sequencing primer corresponding to the base of the nucleic acid template 703 may result in the release of one hydrogen ion as byproduct. Alternatively, the oligonucleotide strand 703 may be cleaved by the cleavage reagent and washed away after the denaturing process, while its complementary oligonucleotide strand 705 may be immobilized on the surface of the reaction wells. The ends of the oligonucleotide strand 705 as well as the capturing oligos 526 and 528 may be blocked by the addition of ddNTPs 509 (see (h) and (i) of FIG. 7). With the addition of another sequencing primer 713, the sequencing of the oligonucleotide strand 705 may be started from its 3′ end. As such, both ends of the nucleic acid template may be sequenced, enabling more accurate sequencing with higher efficiency compared with sequencing from a single-end of the template according to the aforementioned embodiments accompanied by FIG. 4.

As described in the aforementioned embodiments, when the sensor connected with each reaction well is an ISFET sensor, the ion-sensitive layer in the sensor may detect the concentration change of the hydrogen ion. In one embodiment, the release of the hydrogen ion may occur on the surface or in proximity to the ion-sensitive layer, ensuring the sensitivity and accuracy of the detection. On one side, nevertheless, the hydrogen ions may diffuse away from the surface of the ion-sensitive layer. On the other, the 3′ end of the primer strand may have certain distance from the ion-sensitive layer, considering the hybridized double strands may have less flexibility as compared to the single-stranded template. As a result, the sensitivity and accuracy of the detection of the hydrogen ions may be affected.

In one embodiment, with the completion of the template amplification in the reaction wells, a reagent solution containing a plurality of microbeads may be delivered into the reaction chamber by the fluidics delivering unit 104, such that the microbeads may diffuse into the reaction wells. In one embodiment, the microbeads may have a diameter in micrometer or nanometer scale, and may be made of hydrogel, polymers (e.g. polystyrene) or metal (e.g. gold). In one embodiment, each of the reaction wells may include a single microbead which may function as a porous cap to confine the hydrogen ions in proximity to the bottom of the reaction wells. That is, the microbeads may prevent the diffusion of the released hydrogen ions away from the bottom portion of the reaction well, or lateral diffusion into other reaction wells. Further, the microbeads may confine the elongated primer-template duplex in vicinity of the bottom of the reaction wells, such that with the further extension of the primer strand, the released hydrogen ions may be accurately detected by the ion-sensitive layer.

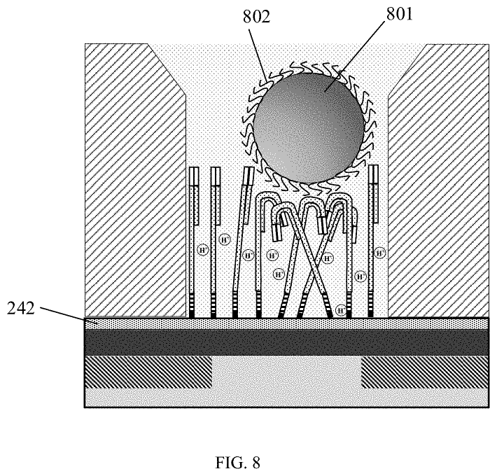

FIG. 8 illustrates the use of microbeads to control ion diffusion in response to the nucleic acid template sequencing according to various embodiments of the present disclosure. In one embodiment, the diameter of the microbead 801 may be adjusted based on the size of the reaction wells, such that a single microbead may be disposed into each of the reaction wells. Additionally, a plurality of single-stranded oligonucleotides 802 may be immobilized on the surface of the microbeads. In one embodiment, the single-stranded oligonucleotides immobilized on the surface of the microbeads may not be annealed with primers, thereby avoiding primer extension reactions which may cause polyclonal reaction in the reaction wells. In some of the optional embodiments, the single-stranded oligonucleotides immobilized on the surface of the microbeads may be annealed with primers with end blocks, thereby avoiding primer extension reactions. Since the bases in the single-stranded oligonucleotides are positively charged, the microbeads may reduce the diffusion of the released hydrogen ions away from the ion-sensitive layer or even out of the reaction well. As such, the released hydrogen ions may be confined in proximity to the ion-sensitive layer. In another embodiment, the surface of the microbeads may be modified with positively-charged polymers, which may also confine the hydrogen ions in the reaction well due to electric repulsion. In other embodiments, the surface of the microbeads may be modified with other biological molecules or chemical polymers, for which the present disclosure will not intend to limit. In one embodiment, the modified molecules on the surface of the microbeads may be inactive, without affecting the primer extension reaction in the sequencing step.

In another embodiment, the parameters of the reaction wells including volume and aspect ratio (e.g. the ratio between the diameter and depth of a reaction well) may be adjusted. For example, the depth of the reaction well may be increased such that the released hydrogen ions due to dNTP incorporation may be confined in the reaction well without lateral diffusion. In another embodiment, the determination of the parameters of the reaction wells may be in accordance with the microbeads disposed into the reaction wells. As such, each reaction well may include a single microbead, while the fluidic communication between the reaction chamber and the opening of the reaction well may not be influenced by the microbeads.

According to the aforementioned embodiments of the present disclosure, the exemplary apparatus and method for nucleic acid sequencing may provide a variety of advantages including high accuracy, efficiency, portability and affordability for users. For example, the digital capture of the nucleic acid templates in the reaction well may result in accurate and efficient amplification of the template to form a high density of nucleic acid template clones in each reaction well, which may further improve the accuracy of the following nucleic acid template sequencing. Furthermore, through the digital capture, the presence or absence of the template in each of the reaction wells may be easily determined through binary signal output. In addition, the time-consuming emulsion PCR may be avoided, such that the efficiency and accuracy of the genetic sequencing may further be improved.

In another embodiment, each of the reaction wells may be connected to an ISFET sensor which may detect the concentration change of the reactors or by-products corresponding to the nucleotide extension reactions. As such, natural reactors including dNTPs may be used in the nucleic acid sequencing apparatus and method, without the requirement of fluorescent-labeled reversible terminators or the use of complex fluorescent imaging platform. The size of the exemplary nucleic acid sequencing apparatus may be reduced, and because of the significant reduce in reagent cost, the sequencing expense per run may be more affordable to users. Furthermore, since the template is immobilized on the bottom portions of the reaction wells close to the ISFET sensor, it may be easier and more efficient for the released hydrogen ions in close vicinity of the ion-sensitive layer, as compared to the template attached to a microbead disposed in the reaction well after emulsion PCR. In one embodiment, the nucleic acid sequencing apparatus and method may achieve a complete process from sample extraction/preparation to completion of sequencing in approximately 190-280 minutes.

Although the principles and implementations of the present disclosure are described by using specific embodiments in the specification, the foregoing descriptions of the embodiments are only intended to help understand the method and core idea of the method of the present disclosure. Meanwhile, a person of ordinary skill in the art may make modifications to the specific implementations and application range according to the idea of the present disclosure. In conclusion, the content of the specification should not be construed as a limitation to the present disclosure.

What is claimed is:

1. A method for nucleic acid sequencing, comprising:
immobilizing at least two capturing oligonucleotides with different sequences in a plurality of reaction wells;
immobilizing a plurality of single-stranded nucleic acid templates in the plurality of reaction wells via annealing between the plurality of single-stranded nucleic acid templates and the at least two capturing oligonucleotides, wherein each of the single-stranded nucleic acid templates includes two regions complementary to the different sequences of the at least two capturing oligonucleotides, respectively;
amplifying the immobilized plurality of single-stranded nucleic acid templates and producing a population of single-stranded nucleic acid template clones on surfaces of the plurality of reaction wells, wherein the population of single-stranded nucleic acid template clones is annealed with a plurality of sequencing primers;
sequentially disposing different types of nucleotide trisphosphates into the plurality of reaction wells wherein the different types of nucleotide trisphosphates are known, and detecting, by one or more ion-sensitive field-effect transistors (ISFETs), an ion concentration change in the plurality of reaction wells in response to incorporation of one of the different types of nucleotide trisphosphates at 3′ end of one of the sequencing primers, when the one of the different types of nucleotide trisphosphates is complementary to a corresponding nucleotide in the population of single-stranded nucleic acid template clones; and
sequencing the population of single-stranded nucleic acid template clones by repeating the sequentially disposing of the different types of nucleotide trisphosphates and the detecting, by the one or more ISFETs, of the ion concentration change in the plurality of reaction wells.

2. The method for nucleic acid sequencing according to claim 1, wherein:
a number of the plurality of single-stranded nucleic acid templates immobilized on a surface of each of the reaction wells via the annealing is less than or equal to a pre-determined value, wherein the pre-determined value is one.

3. The method for nucleic acid sequencing according to claim 1, wherein the amplifying the immobilized plurality of single-stranded nucleic acid templates and producing the population of single-stranded nucleic acid template clones on the surfaces of the plurality of reaction wells further comprises:
amplifying the immobilized plurality of single-stranded nucleic acid templates, thereby generating a plurality of double-stranded nucleic acid template clones;
denaturing the plurality of double-stranded nucleic acid template clones; and
producing a population of single-stranded nucleic acid template clones on the surface of the plurality of reaction wells.

4. The method for nucleic acid sequencing according to claim 1, wherein:
a total number of the plurality of single-stranded nucleic acid templates disposed into the plurality of reaction wells is less than or equal to a total number of the plurality of reaction wells.

5. The method for nucleic acid sequencing according to claim 4, wherein:
the total number of the plurality of nucleic acid templates disposed into the plurality of reaction wells is less than or equal to 70% of the total number of the plurality of reaction wells.

6. The method for nucleic acid sequencing according to claim 1, further comprising:
determining a loading rate of the plurality of reaction wells, wherein the load rate includes a ratio between a number of the reaction wells containing the immobilized single-stranded nucleic acid templates and a total number of the plurality of reaction wells.

7. The method for nucleic acid sequencing according to claim 1, further comprising:
repeating the step of disposing the plurality of single-stranded nucleic acid templates into the plurality of reaction wells and the step of amplifying the immobilized plurality of single-stranded nucleic acid templates.

8. The method for nucleic acid sequencing according to claim 5, wherein:
a loading rate is determined after each loading cycle including the step of disposing the plurality of single-stranded nucleic acid templates into the plurality of reaction wells and the step of amplifying the immobilized plurality of single-stranded nucleic acid templates.

9. The method for nucleic acid sequencing according to claim 1, further comprising the step of:
disposing a plurality of microbeads into the plurality of reaction wells, wherein a surface of the plurality of microbeads is attached with single-stranded oligonucleotides or polymers.

10. The method for nucleic acid sequencing according to claim 9, wherein:
the step of disposing the plurality of microbeads into the plurality of reaction wells is concurrently with, or before the step of detecting, by the one or more ISFETs, ion concentration change in the plurality of reaction wells.

11. A method for producing single-stranded nucleic acid template clones on a reaction well array, comprising:
providing the reaction well array including a plurality of reaction wells, wherein at least two capturing oligonucleotides with different sequences are immobilized on a surface of each of the reaction wells;
adding a solution including a plurality of single-stranded nucleic acid templates into the plurality of reaction wells, wherein:
each of the single-stranded nucleic acid templates includes two regions complementary to the different sequences of the at least two capturing oligonucleotides, respectively,
the plurality of single-stranded nucleic acid templates is immobilized on the surface of the plurality of reaction wells via annealing between the nucleic acid templates and the at least two capturing oligonucleotides, and
a number of the single-stranded nucleic acid templates immobilized on the surface of each the plurality of reaction wells via the annealing is less than or equal to a pre-determined value; and
amplifying the immobilized plurality of single-stranded nucleic acid templates, thereby generating a plurality of double-stranded nucleic acid template clones; and
denaturing the plurality of double-stranded nucleic acid template clones and producing a population of single-stranded nucleic acid template clones on the surface of the plurality of reaction wells.

12. The method for producing the single-stranded nucleic acid template clones on the reaction well array according to claim 11, wherein:
the pre-determined value is one.

13. The method for producing the single-stranded nucleic acid template clones on the reaction well array according to claim 11, wherein:
a total number of the plurality of nucleic acid templates in the solution is less than or equal to a total number of the plurality of reaction wells.

14. The method for producing the single-stranded nucleic acid template clones on the reaction well array according to claim 13, wherein:
the total number of the plurality of nucleic acid templates in the solution is less than or equal to 70% of the total number of the plurality of reaction wells.

15. The method for producing the single-stranded nucleic acid template clones on the reaction well array according to claim 11, further comprising:
determining a loading rate of the plurality of reaction wells, wherein the load rate includes a ratio between a number of the reaction wells containing the immobilized plurality of nucleic acid templates and a total number of the plurality of reaction wells.

16. The method for producing the single-stranded nucleic acid template clones on the reaction well array according to claim 11, further comprising:
repeating the step of disposing the plurality of nucleic acid templates into the plurality of reaction wells and the step of amplifying the immobilized plurality of nucleic acid templates.

17. The method for producing the single-stranded nucleic acid template clones on the reaction well array according to claim 15, wherein:
the loading rate is determined by measuring, by one or more ion-sensitive field-effect transistors (ISFETs) configured to provide at least one output signal in response to a concentration or presence of one or more ions proximate thereto, ion concentration change corresponding to the amplification of the immobilized plurality of nucleic acid templates in the reaction wells, wherein the plurality of reaction wells is associated with the one or more ISFETs.

18. An apparatus for nucleic acid sequencing, the apparatus comprising:
a sensor array, including a plurality of ion-sensitive field-effect transistors (ISFETs) configured to provide at least one output signal corresponding to a concentration or presence of one or more ions proximate thereto;
a flow cell including an input, an output and a flow chamber, wherein the flow chamber is in fluidic connection with an opening of each reaction well of an array of reaction wells, wherein at least two capturing oligonucleotides with different sequences are immobilized on a surface of each of the reaction wells, and the different sequences of the at least two capturing oligonucleotides are complementary to two regions of a to-be-sequenced nucleic acid template; and
a fluidics delivering unit, configured to be in fluidic connection with the input of the flow cell, and configured to deliver at least one of the to-be-sequenced nucleic acid template and different types of known nucleotide trisphosphates, in a direction from the input to the output, to the reaction chamber, wherein:
each of the reaction wells is associated with one of the plurality of ISFETs in the sensor array, and the one of the plurality of ISFETs is configured to provide the at least one output signal in response to ion concentration change in each of the reaction wells, and
the ion concentration change corresponds to incorporation of one of the different types of nucleotide trisphosphates at 3′ end of a sequencing primer annealed to the to-be-sequenced nucleic acid template, when the one of the different types of nucleotide trisphosphates is complementary to a corresponding nucleotide in the to-be-sequenced nucleic acid template.

19. The apparatus for nucleic acid sequencing according to claim 18, wherein:
the fluidics delivering unit is further configured to deliver a plurality of microbeads into the reaction chamber, such that the plurality of microbeads control diffusion of ionic byproducts generated from the incorporation of one of the different types of nucleotide trisphosphates at the 3′ end of the sequencing primer.

20. The apparatus for nucleic acid sequencing according to claim 18, wherein:
the sensor array and the array of reaction wells are integrated on a same semiconductor chip.
